(19) United States
(12) Patent Application Publication
(10) Pub. No.: US 2005/0036989 A1
(43) Pub. Date: Feb. 17, 2005

(54) Subgroup B adenoviral vectors for treating disease

(75) Inventors: Jerry Yuqiao Shen, Orinda, CA (US); Annie Shen, Richmond, CA (US); Aleida Perez, Alameda, CA (US); Elizabeth Sevilla, San Jose, CA (US); Amy Aspelund, Crockett, CA (US)

Correspondence Address: ONYX PHARMACEUITICALS, INC., 3031 RESEARCH DRIVE, RICHMOND, CA, 94806, US

(73) Assignee: Onyx Pharmaceuticals, Inc., Richmond, CA (US)

(21) Appl. No.: 10/874,049

(22) Filed: Jun. 22, 2004

Related U.S. Application Data
(60) Provisional application No. 60/488,678, filed on Jul. 18, 2003.

(51) Int. Cl. A61K 48/00; C12N 15/86

(52) U.S. Cl. 424/93.2; 435/456

(57) ABSTRACT

Methods and compositions for treating disease using human subgroup B adenovirus, vectors derived from such viruses, including expression vector systems in which one or more subgroup B adenoviral genes are replaced by a foreign gene.

### FIELD OF THE INVENTION

The invention described herein relates to the field of treating disease using human subgroup B adenoviruses.

### BACKGROUND OF THE INVENTION

Conditionally replicating viruses represent a promising new class of anti-cancer agents. Derivatives of human adenovirus type 5 (Ad5) have been developed that selectively replicate in, and kill, cancer cells. The prototype of such viruses, ONYX-015, a subgroup C adenovirus, has demonstrated encouraging results in several phase I and phase II clinical trials with patients having recurrent head-and-neck cancer, and patients having liver metastatic disease.

In order for adenovirus to replicate efficiently in cells, the adenoviral E1b gene product, p55, forms a complex with the host cell p53 protein, thereby sequestering and/or inactivating p53 and producing a cell that is deficient in p53 function. Such a cell made deficient in p53 function can support replication of the adenovirus. In this way, wild-type adenovirus is able to replicate in cells containing p53, as the adenovirus p55 proteins inactivates and/or sequesters the host cell p53 protein. Onyx-015 is a recombinant adenovirus comprising an E1b locus encoding a mutant p55 protein that is substantially incapable of forming a functional complex with p53 protein in infected cells when it is administered to an individual or cell population comprising a neoplastic cell capable of being infected by the recombinant adenovirus. The substantial incapacity of the recombinant adenovirus to effectively sequester p53 protein in infected non-neoplastic cells results in the introduced recombinant adenoviral polynucleotide(s) failing to express a replication phenotype in non-neoplastic cells. By contrast, neoplastic cells which lack a functional p53 protein support expression of a replication phenotype by the introduced recombinant adenovirus which leads to ablation of the neoplastic cell by an adenoviral cytopathic effect and/or expression of a negative selection gene linked to the replication phenotype.

A lesson learned from the on-going clinical trials with Onyx-015 is that efficacy rather than toxicity appears to limit its therapeutic benefit. To date, no dose-limiting toxicity has been observed. One strategy to enhance the clinical efficacy of oncolytic viruses is to combine them with other therapies, eg. standard chemotherapy, or to arm them with anti-cancer genes, such as anti-angiogenesis factors, cytotoxic agents, pro-drug converting enzymes, or cytokines, etc. Another approach, which can be combined with chemotherapy and anti-cancer genes, is to genetically alter the virus to render it more potent, i.e. replicate faster, produce more viral progenies, and enhance tissue and/or cell type specificity etc. The goal here being to generate therapeutic viruses that kill cancer cells more rapidly, selectively, and eventually eradicate the cancer.

A critical aspect of such therapeutic strategies depends on the ability of adenovirus to enter target cells. This process is a multi-step event believed to be initiated by attachment of the virus to cells by binding of the adenovirus fiber-knob protein to its cellular receptor CAR (Bergelson et al. (1997) Science 275: 1320-1323). In a second step, internalization of the virus is then mediated by αvβ₃ and αvβ₅ integrins through interaction with the RGD-domain of the adenovirus penton base (Wickham et al. (1993) Cell 73: 309-319; Mathias et al. (1994) J. Virol. 68: 6811-6814). After internalization, the virus particles travel to the nuclear membrane. During this process, the capsid is removed and finally the viral DNA is released into the nucleus where replication of viral DNA is initiated. The presence of CAR on cells appears to be a major determining factor for the efficacy of adenovirus infection. In contrast, there is no association with the expression of secondary adenovirus receptors, including αvβ₃ and αvβ₅ integrins (Hemmi et al. (1998) Hum Gene Ther. 9: 2363-2373).

A potential drawback to using subgroup C adenovirus to treat cancer is two fold. First, recent experimental work has shown that CAR expression is reduced in tumor cells compared to normal cells both in vitro and in vivo in patients suffering from certain forms of cancer. It was found by RT-PCR and Western blot analysis that there is a good correlation between the level of CAR expression and the transfection efficiency adenovirus. (Jee Y S, Lee S G, Lee J C, Kim M J, Lee J J, Kim D Y, Park S W, Sung M W, Heo DS. Anticancer Res 2002 Sep-Oct;22(5):2629-34). Also, transfection of CAR into human bladder carcinoma cells with no endogenous CAR expression increased infectibility of these cells significantly (Li et al. (1999) Cancer Res. 59: 325-330).

A second drawback associated with using subgroup C adenovirus for cancer therapy is the presence of CAR in hepocytes that has the undesirable side effect of faciliting the accumulation of the virus in the liver. Subgroup B viruses which subgroup B system and optimal tandem fiber system demonstrate reduced liver transduction by over 2 logs compared to an Ad5 fiber vector Schoggins J W, Gall J G, Falck-Pedersen E. J Virol 2003 January;77(2):1039-48.

As mentioned above, the prototype oncolytic adenovirus is Onyx 015, which is a subgroup C virus. For the reasons discussed above, adenoviral vectors constructed from subgroup C viruses have certain properties that limit their oncolytic potential. To provide the physican with another oncolytic virus, it would be beneficial to produce an adenovirus that has the properies of Onyx 015, and the properties of subgroup B adenoviruses.

There are reports in the scientific and patent literature that describe genetic aspects of subgroup B adenoviruses, including nucleotide sequences of certain regions of these viruses.

WO0240693A1 shows adenoviral replicon comprises a recombinant adenovirus with a fusion between DNA from Ad5 and subgroup B adenoviral DNA.

WO0240665 shows a packaging cell line capable of complementing recombinant adenoviruses based on serotypes from subgroup B, preferably adenovirus type 35.

WO0227006 shows a means and methods for transduction of a skeletal muscle cell

use of a gene delivery vehicle derived from an adenovirus, having a tropism for said cells. The gene delivery vehicle comprises at least a tropism determining part of an adenoviral fiber protein of subgroup B

WO0052186 describes an adenovirus subgroup B nucleic acid delivery vehicle with a tissue tropism for fibroblast-like or macrophage-like cells.

WO0031285 provides a nucleic acid delivery vehicle with a tissue tropism for smooth muscle cells and/or endothelial cells. In one aspect the nucleic acid delivery vehicle is a virus capsid of a subgroup B adenovirus.

WO8906282 describes a functional mutated E1A gene of human adenovirus subgroup B:1 having a modified autorepression functional domain.

U.S. Pat. No. 6,492,169 presents a packaging cell line to complement recombinant adenoviruses based on serotypes from subgroup B, preferably adenovirus type 35.

U.S. Pat. No. 5,770,442 shows a recombinant adenovirus comprising a subgroup B adenoviral chimeric fiber protein

U.S. No. 4,920,211 shows a functional mutated E1A gene of human adenovirus subgroup B:1 which has a modified autorepression functional domain that is effective to express E1 A products that stimulate without net repression of promoters controlling an E1 A mutated gene

### BRIEF DESCRIPTION OF THE DRAWINGS

FIG. 1 shows the complete nucleotide sequence of human subgroup B adenovirus type-3 and the region that encodes the E1B55K protein.

FIG. 2 shows the complete nucleotide sequence of human subgroup B adenovirus type-34 and the region that encodes the E1B55K protein.

FIG. 3 shows the cDNA nucleotide sequence of the E1A region of human subgroup B adenovirus type-3.

FIG. 4 shows the amino acid sequence of the E1A region encoded by the cDNA of human subgroup B adenovirus type-3.

FIG. 5 shows the cDNA nucleotide sequence of the E1A region of human subgroup B adenovirus type-34.

FIG. 6 shows the amino acid sequence of the E1A region encoded by the cDNA of human subgroup B adenovirus type-34.

FIG. 7 shows the DNA sequence of open reading frame 6 of human subgroup B adenovirus type-3.

### SUMMARY OF THE INVENTION

A feature of the present invention is the description of recombinant, oncolytic human subgroup B adenoviruses.

The invention also presents the full genomic sequences of human subgroup B adenoviruses types 3 and 34.

In another aspect, the invention includes the use of recombinant, human subgroup B adenoviruses, and recombinant viral vectors derived therefrom for the expression of a heterogenous DNA sequence.

Another embodiment of the present invention relates to human adenovirus expression vector systems based on subgroup B types 3 and 34 in which part, or all of one or both of the E1 and E3 gene regions are deleted.

A feature of the present invention is the description of recombinant, oncolytic human subgroup B adenoviruses that lack an expressed viral oncoprotein capable of binding a functional tumor suppressor gene product, and that infect cells primarily by a CAR indepenent mechanism.

Another feature of the present invention is the description of an oncolytic human subgroup B adenovirus that lacks an expressed viral oncoprotein capable of binding a functional tumor suppressor gene product.

Another aspect of the invention relates to human subgroup B adenoviruses which lack the ability to encode a functional E1A or E1B 55k viral oncoprotein.

A further aspect of the invention is a description of treating disease using recombinant, human subgroup B adenoviruses.

These and other aspects of the invention will become apparent to a skilled practitioner of this field upon a full consideration of the following.

### DETAILED DESCRIPTION OF THE INVENTION

All publications and patent applications cited throughout this patent are incorporated by reference to the same extent as if each individual publication or patent/patent application is specifically and individually indicated to be incorporated by reference in their entirety.

The practice of the present invention will employ, unless otherwise indicated, conventional microbiology, immunology, virology, molecular biology, and recombinant DNA techniques which are within the skill of the art. These techniques are fully explained in the literature. See, eg., Maniatis et al., Molecular Cloning: A Laboratory Manual (1982); DNA Cloning: A Practical Approach, vols. I & II (D. Glover, ed.); Oligonucleotide Synthesis (N. Gait, ed. (1984)); Nucleic Acid Hybridization (B. Hames & S. Higgins, eds. (1985)); Transcription and Translation (B. Hames & S. Higgins, eds. (1984)); Animal Cell Culture (R. Freshney, ed. (1986)); Perbal, A Practical Guide to Molecular Cloning (1984). Sambrook et al., Molecular Cloning: A Laboratory Manual (2.sup.nd Edition); vols. I, II & III (1989). See also, Hermiston, T. et al., Methods in Molecular Medicine: Adenovirus Methods and Protocols, W. S. M. Wold, ed, Humana Press, 1999.

A. Definitions

Unless defined otherwise, all technical and scientific terms used herein have thesame meaning as commonly understood by one of ordinary skill in the art to which this invention belongs. Although any methods and materials similar or equivalent to those described herein can be used in the practice or testing of the present invention, the preferred methods and materials are described.

The definitions set forth in U.S. Pat. Nos. 5,677,178, and 5,801,029 are applicable here and include the following terms.

“Replication deficient virus” refers to a virus that preferentially inhibits cell proliferation in a predetermined cell population (e.g., cells substantially lacking p53, and/or RB function) which supports expression of a virus replication phenotype, and which is substantially unable to inhibit cell proliferation, induce apoptosis, or express a replication phenotype in cells comprising normal p53 or RB levels characteristic of non-replicating, non-transformed cells. Typically, a replication deficient virus exhibits a substantial decrease in plaquing efficiency on cells comprising normal p53 or RB function.

As used herein, the term “p53 function” refers to the property of having an essentially normal level of a polypeptide encoded by the p53 gene (i.e., relative to non-neoplastic cells of the same histological type), wherein the p53 polypeptide is capable of binding an E1b p55 protein of subgroup C wild-type adenovirus 34. For example, p53 function may be lost by production of an inactive (i.e., mutant) form of p53 or by a substantial decrease or total loss of expression of p53 polypeptide(s). Also, p53 function may be substantially absent in neoplastic cells which comprise p53 alleles encoding wild-type p53 protein; for example, a genetic alteration outside of the p53 locus, such as a mutation that results in aberrant subcellular processing or localization of p53 (e.g., a mutation resulting in localization of p53 predominantly in the cytoplasm rather than the nucleus), or the loss or inactivation of a molecule by which p53 acts, can result in a loss of p53 function. That is, there may be an alteration in the biochemical pathway by which p53 acts, which would cause a loss of p53 function.

As used herein, the term “replication phenotype” refers to one or more of the following phenotypic characteristics of cells infected with a virus such as a replication deficient adenovirus: (1) substantial expression of late gene products, such as capsid proteins (e.g., adenoviral penton base polypeptide) or RNA transcripts initiated from viral late gene promoter(s), (2) replication of viral genomes or formation of replicative intermediates, (3) assembly of viral capsids or packaged virion particles, (4) appearance of cytopathic effect (CPE) in the infected cell, (5) completion of a viral lytic cycle, and (6) other phenotypic alterations which are typically contingent upon abrogation of p53 function in non-neoplastic cells infected with a wild-type replication competent DNA virus encoding functional oncoprotein(s). A replication phenotype comprises at least one of the listed phenotypic characteristics, preferably more than one of the phenotypic characteristics.

The term “antineoplastic replication deficient virus” is used herein to refer to a recombinant virus which has the functional property of inhibiting development or progression of a neoplasm in a human, by preferential cell killing of infected neoplastic cells relative to infected nonreplicating, non-neoplastic cells of the same histological cell type.

As used herein, “neoplastic,” “neoplasia,” “cancer,” or “tumor” refer to cells which exhibit relatively autonomous growth, so that they exhibit an aberrant growth phenotype characterized by a significant loss of control of cell proliferation.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, “physiological conditions” refers to an aqueous environment having an ionic strength, pH, and temperature substantially similar to conditions in an intact mammalian cell or in a tissue space or organ of a living mammal. Typically, physiological conditions comprise an aqueous solution having about 150 mM NaCl (or optionally KCl), pH-6.5-8.1, and a temperature of approximately 20.degree.-45.degree. C. Generally, physiological conditions are suitable binding conditions for intermolecular association of biological macromolecules. For example, physiological conditions of 150 mM NaCl, pH 7.4, at 37.degree. C. are generally suitable.

A DNA “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, viral DNA, and even synthetic DNA sequences. A polyadenylation signal and transcription termination sequence will usually be located 3′ to the coding sequence.

A “transcriptional promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background.

DNA “control sequences” refer collectively to promoter sequences, ribosome binding sites, splicing signals, polyadenylation signals, transcription termination sequences, upstream regulatory domains, enhancers, translational termination sequences and the like, which collectively provide for the transcription and translation of a coding sequence in a host cell.

A coding sequence or sequence encoding is “operably linked to” or “under the control of” control sequences in a cell when RNA polymerase will bind the promoter sequence and transcribe the coding sequence into mRNA, which is then translated into the polypeptide encoded by the coding sequence.

A “host cell” is a cell which has been transformed, or is capable of transformation, by an exogenous DNA sequence.

Two polypeptide sequences are “substantially homologous” when at least about 80% (preferably at least about 90%, and most preferably at least about 95%) of the amino acids match over a defined length of the molecule.

Two DNA sequences are “substantially homologous” when they are identical to or not differing in more that 40% of the nucleotides, preferably not more than about 30% of the nucleotides (i.e. at least about 70% homologous) more preferably about 20% of the nucleotides, and most preferably about 10% of the nucleotides.

DNA sequences that are substantially homologous can be identified in a Southern hybridization experiment under, for example, stringent conditions, as defined for that particular system. Highly stringent conditions would include hybridization to filter-bound DNA in 0.5M NaHPO4, 7% sodium dodecyl sulfate (SDS), 1 mM EDTA at 65° C., and washing in 0.1.times SSC/0.1% SDS at 68° C. (Ausubel F. M. et al., eds., 1989, Current Protocols in Molecular Biology, Vol. 1, Green Publishing Associates, Inc., and John Wiley & Sons, Inc., New York, at p. 2.10.3. Defining appropriate hybridization conditions is within the skill of the art. See, e.g., Maniatis et al., supra; DNA Cloning, vols. I & II, supra; Nucleic Acid Hybridization, supra.

A “heterologous” region of a DNA construct is an identifiable segment of DNA within or attached to another DNA molecule that is not found in association with the other molecule in nature.

“Fusion protein” is usually defined as the expression product of a gene comprising a first region encoding a leader sequence or a stabilizing polypeptide, and a second region encoding a heterologous protein. It involves a polypeptide comprising an antigenic protein fragment or a full length adenoviral protein sequence as well as (a) heterologous sequence(s), typically a leader sequence functional for secretion in a recombinant host for intracellularly expressed polypeptide. An antigenic protein fragment is usually about 5-7 amino acids in length.

“Recombinant” polypeptides refers to polypeptides produced by recombinant DNA techniques.

A “substantially pure” protein will be free of other proteins, preferably at least 10% homogeneous, more preferably 60% homogeneous, and most preferably 95% homogeneous.

By “infectious” is meant having the capacity to deliver the adenoviral genome into cells.

“CAR” refers to the receptor on cells which subgroup C adenovirus binds to in the process of infecting and gaining entry to a host cell. It is an acronym for Coksakie Adenovirus Receptor.

“Oncolytic” refers to the ability of the invention human subgroup B adenoviruses to kill neoplastic cells with substantial selectivity over normal cells; that is, while substantially sparing normal cells.

B. General Methods

Adenoviral subgroup B genomes/Coding regions: Human subgroup B adenoviral genomes can be obtained from the American Type Culture Collection (ATCC). The viruses, preferrably from subgroup B types 3 and 34, can be propagated using materials and methods well known in the art, including A549 cells and standard infection and growth techniques. Hermiston, T. et al., Methods in Molecular Medicine: Adenovirus Methods and Protocols, W. S. M. Wold, ed, Humana Press, 1999. Virus can be purified by any number of techiniques including cesium chloride gradient banding centrifugation. See, for example, U.S. Pat. No. 5,837,520 and U.S. Pat. No. 6,008,036.

Viral DNA is prepared for sequening by lysing the virus particles in a lysis solution, preferrably consisting of: 10 mM Tris-HCl (pH8.0), 5 mM EDTA, 0.6% SDS and 1.5 mg per ml of pronase (Sigma Corporation). The solution is preferrably at 37° C. Lysed viral particles are extracted with phenol/chloroform, and viral DNA is precipitated with ethanol. Purified viral DNAs are dissolved in distilled water and used for DNA sequencing.

Next, viral DNAs from either adenovirus subgroup B types 3, or 34, are subjected to limit digestion with an appropriate restriction enzyme, preferrably Sau 3AI, followed by resolving the digested DNAs in a 1% agarose gel. Fragments between 0.8 kb and 1.2 kb in size are purified using a commercial DNA gel extraction kit (Qiagen Corporation), and subsequently cloned into an appropriate vector previously digested with a compatible restriction enzyme. As described more in the Examples, Bam HI can be used to digest the vector, pGem-7zf(+) (Promega Corporation).

Next, several hundred individual clones are sequenced using an automated sequencer, CEQ20000XL (Beckman), and using standard T7 and SP6 Sequencing primers. Contigs were constructed using SeqMan Software (DNAStar Inc.). Based on the constructed sequences, oligonucleotides are synthesized and primer walking can be performed until all contiges are joined. As described below, most regions were covered by at least 2 independent sequencings.

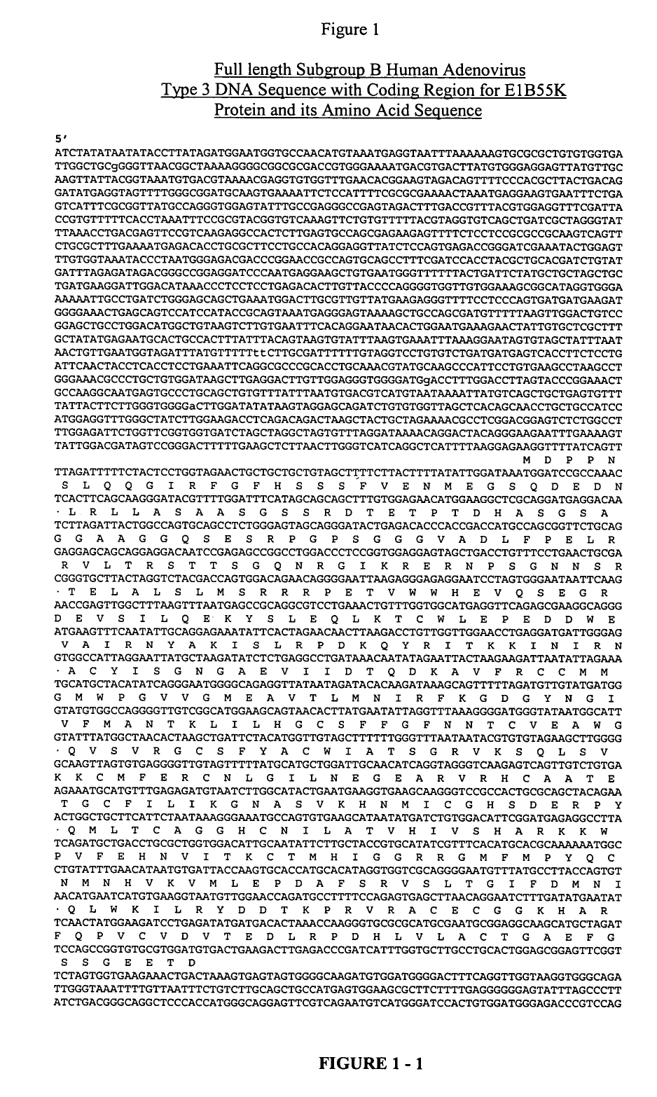

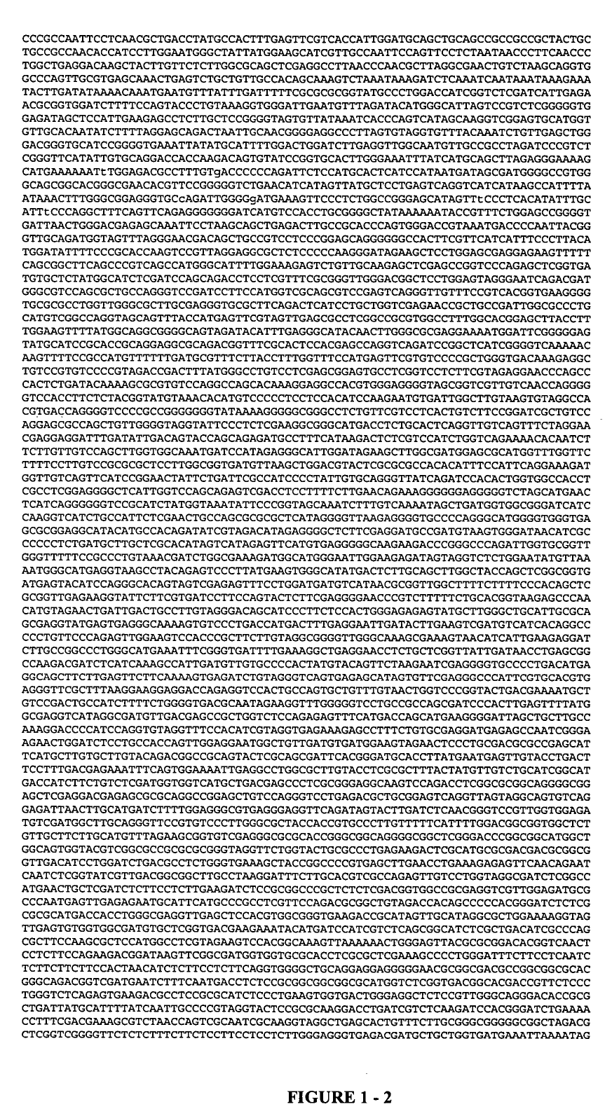

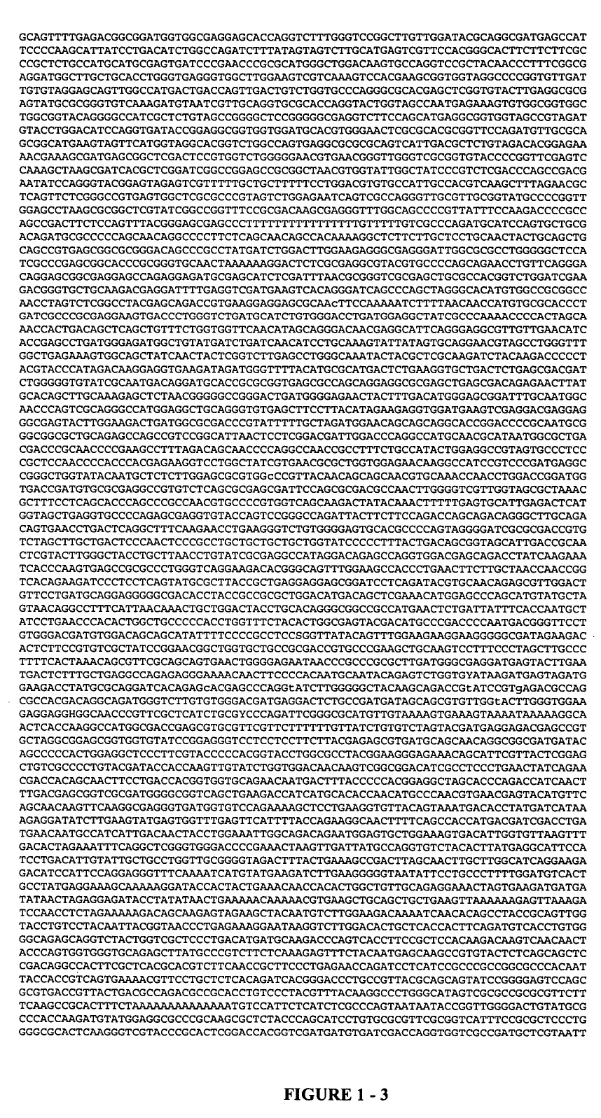

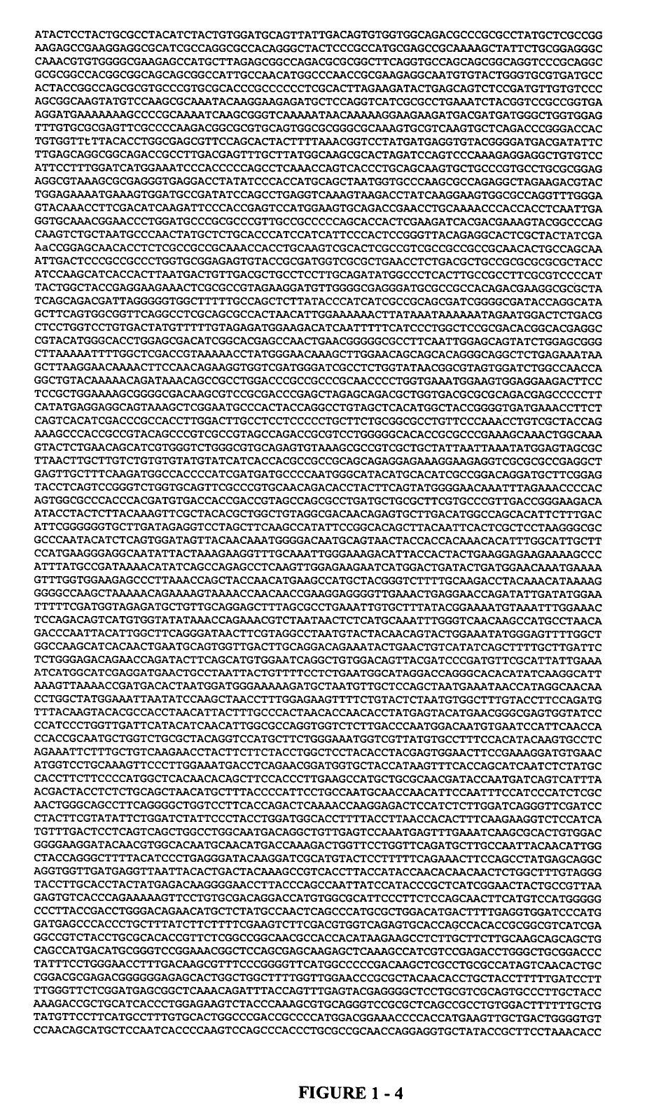

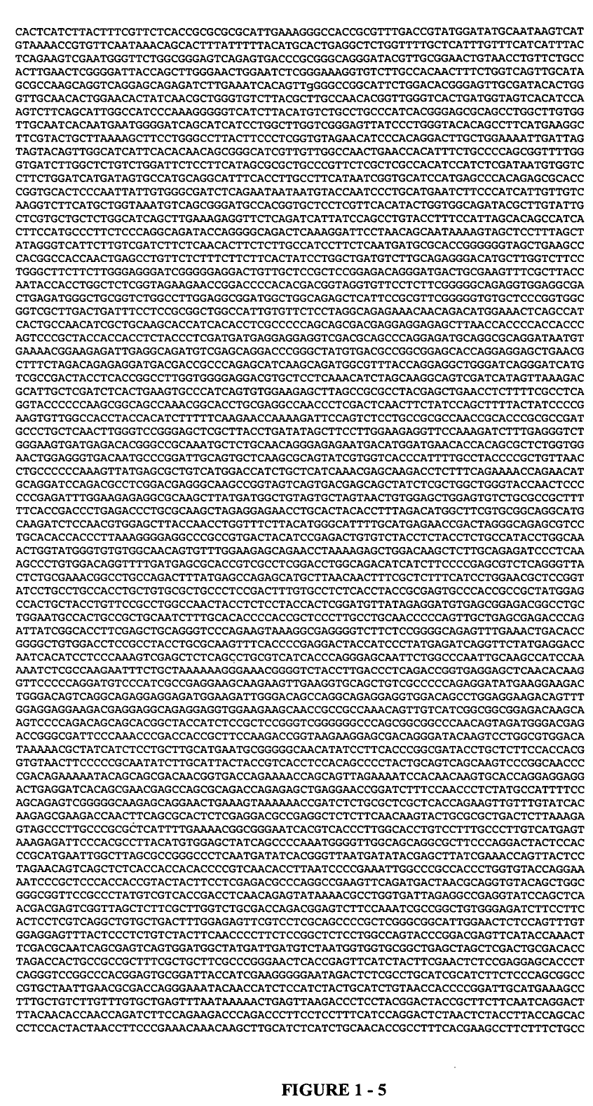

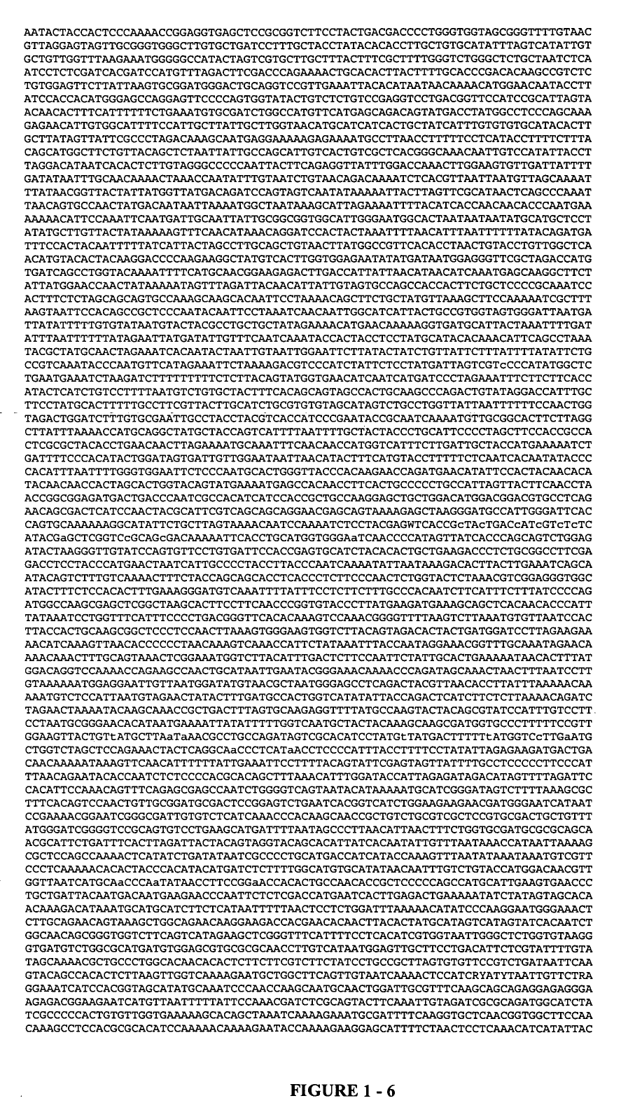

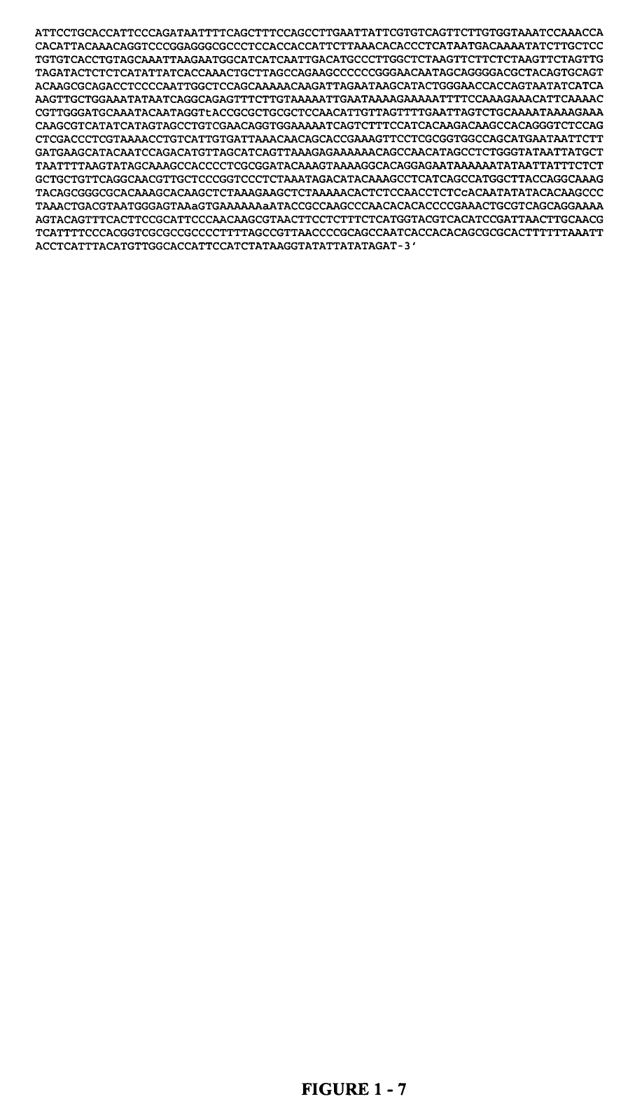

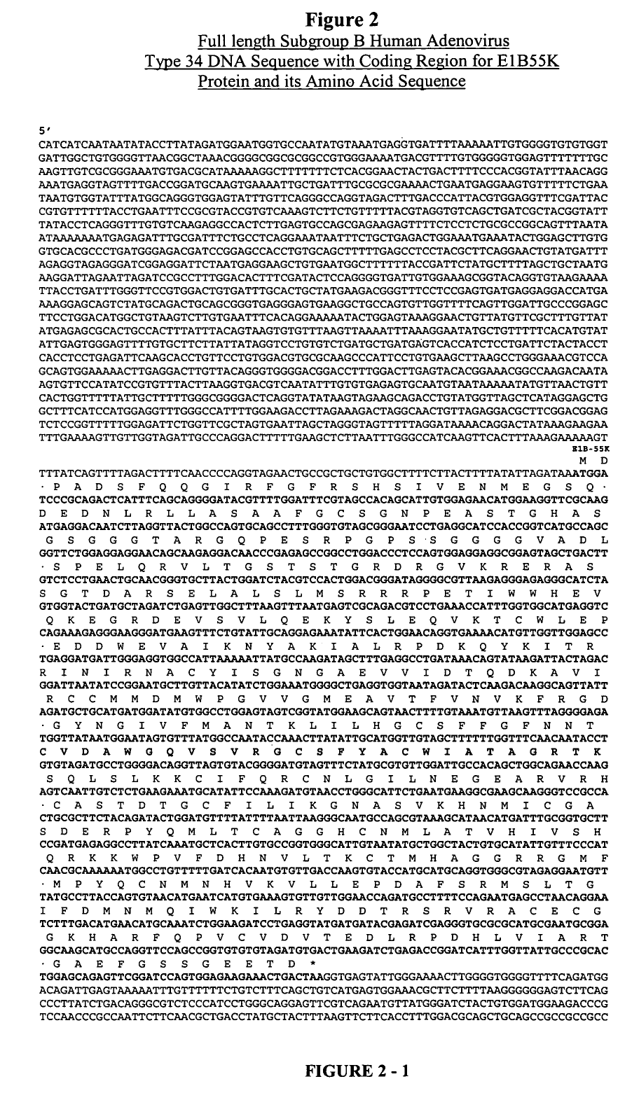

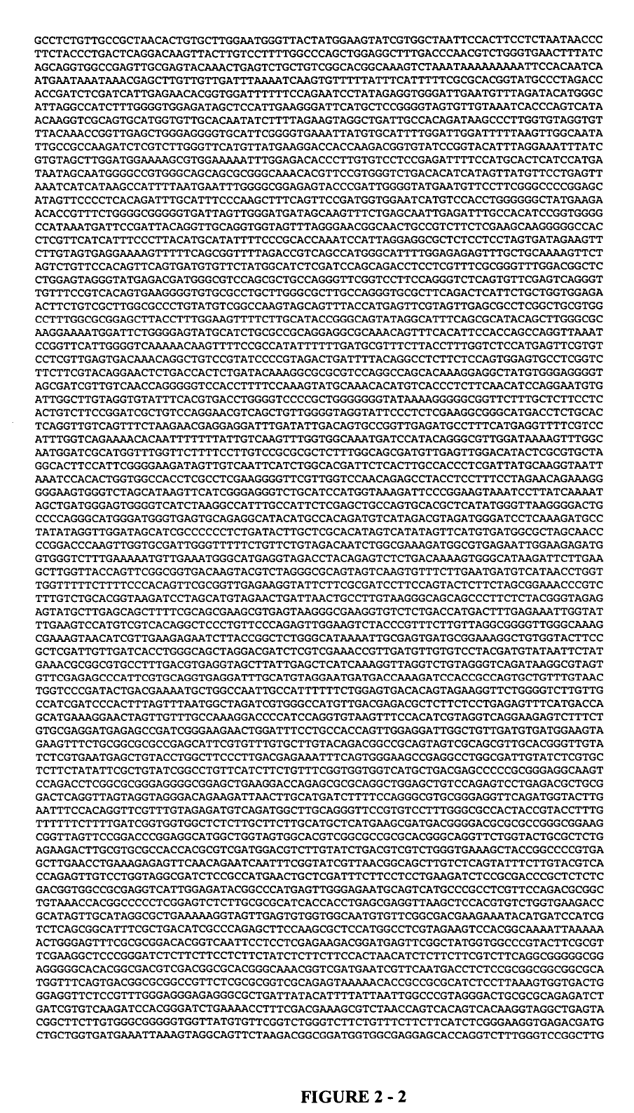

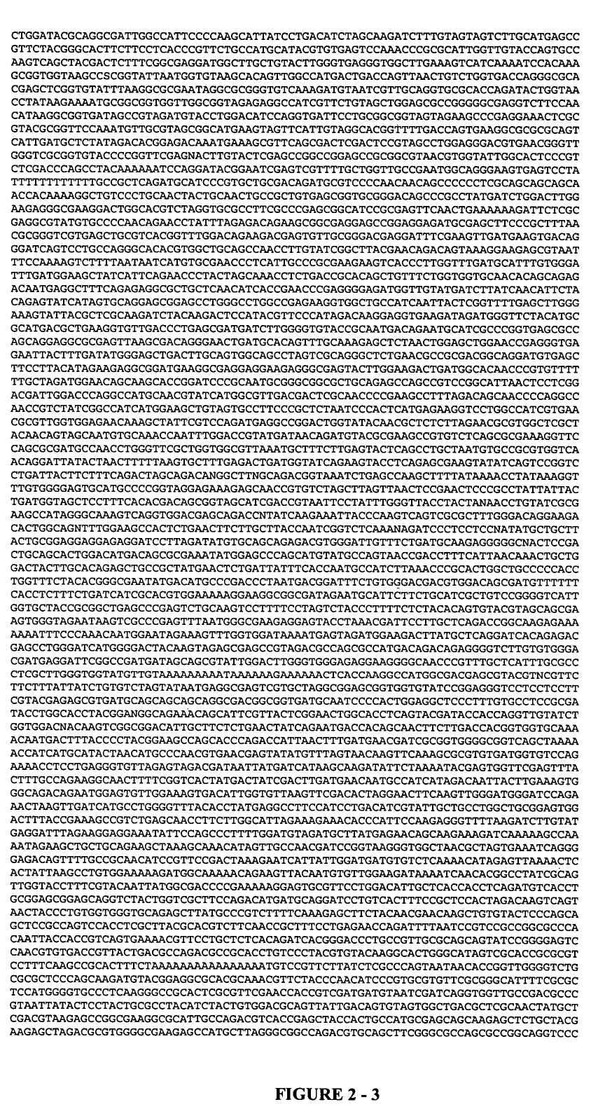

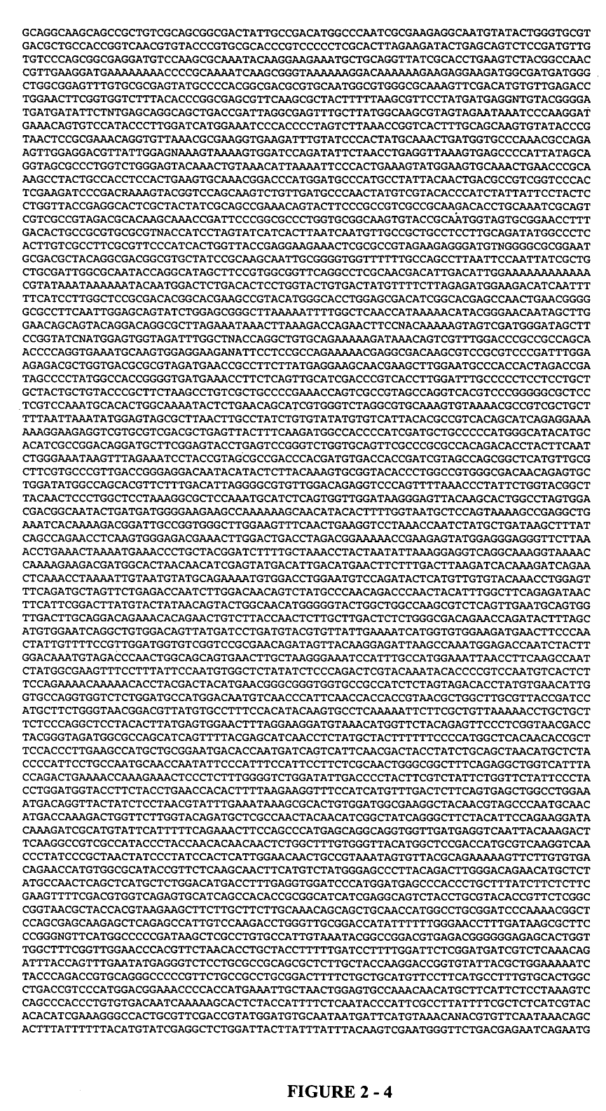

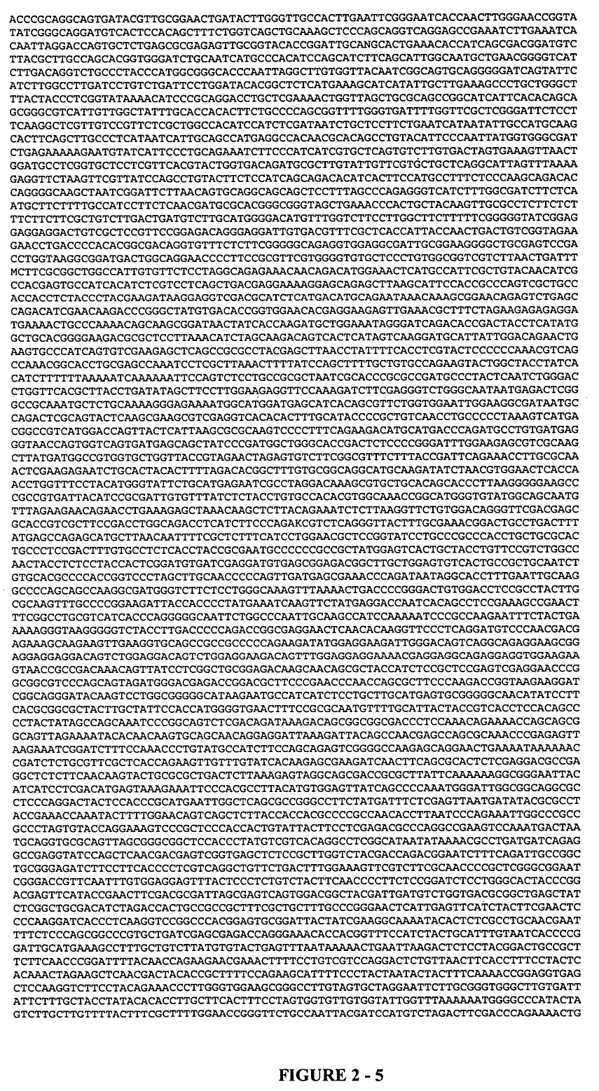

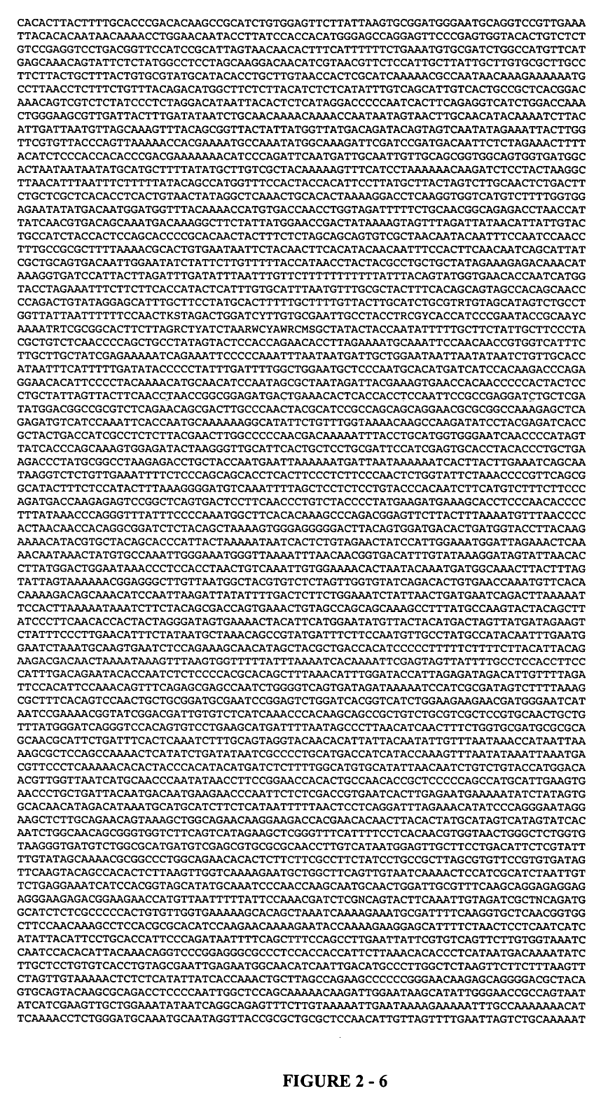

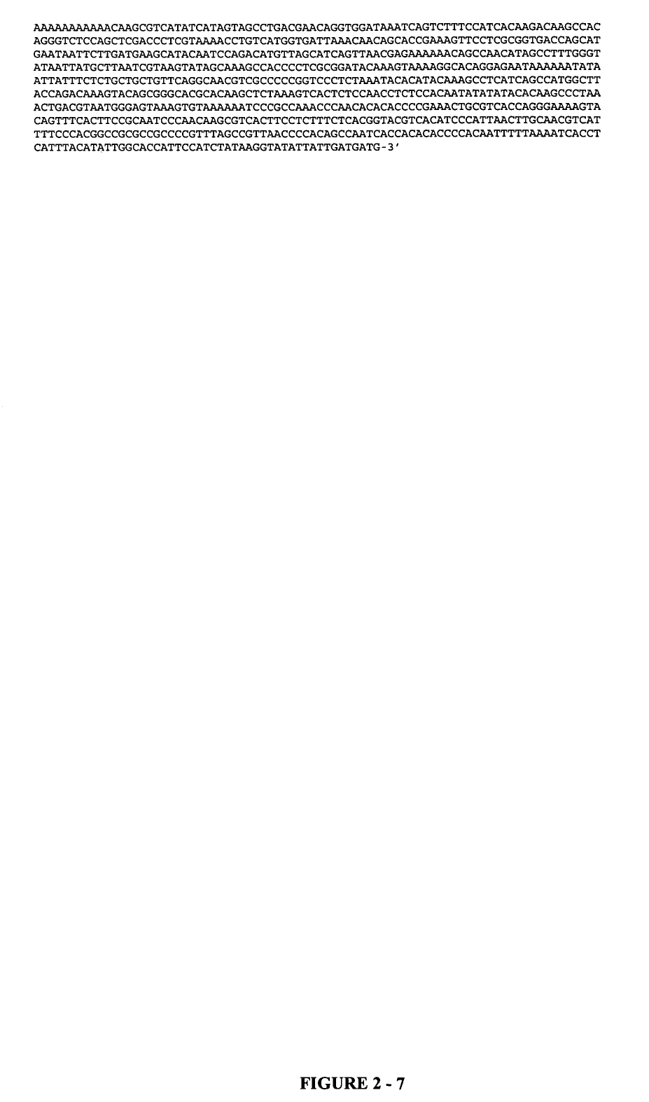

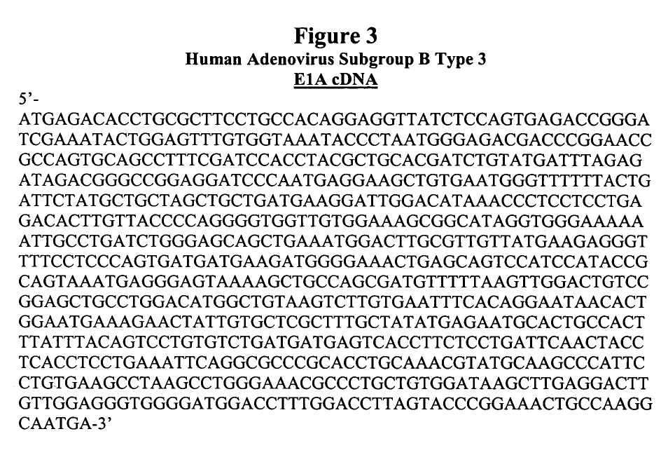

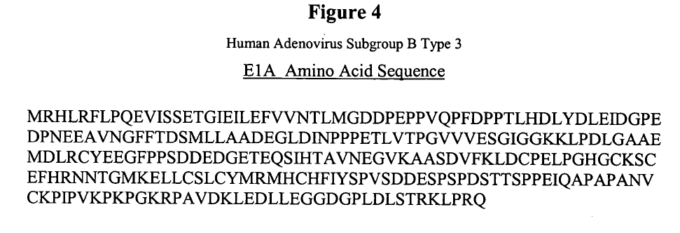

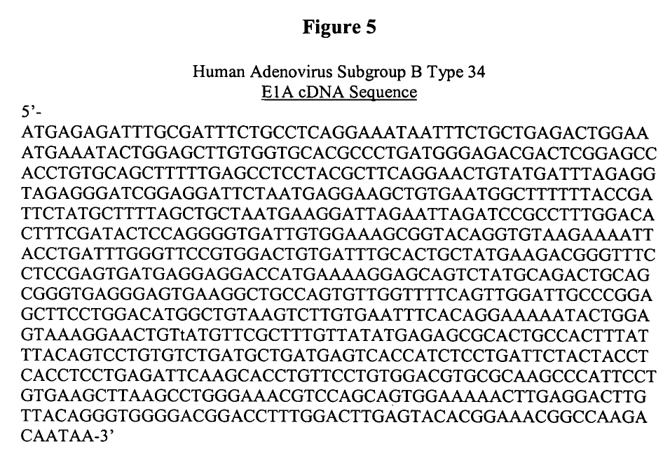

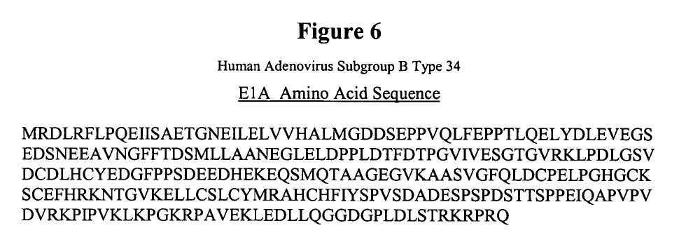

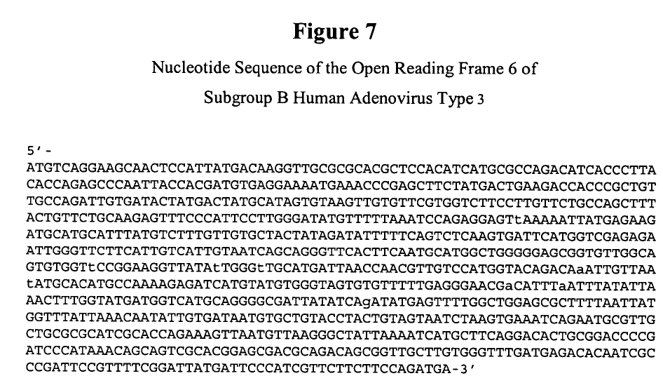

The present invention discloses the complete nucleotide genomic sequences of the human subgroup B adenoviruses types 3 and 34. See FIG. 1 and FIG. 2, respectively. Also shown are the nucleotide sequences for certain regions of these viruses, including E1A (FIGS. 3 and 5, for types 3 and 34, respectively) the amino acid sequences for the E1A regions (FIGS. 4 and 6, for types 3 and 34, respectively). The nucleotide sequences that code for the E1 B region that encodes the 55K protein, and their amino acid sequences, are shown in FIG. 1 and FIG. 2 for adenoviruses types 3 and 34, respectively.

In addition to the genomic sequences of human subgroup B adenovirus types 3 and 34, various regions of these viruses were sequenced, and the amino acid sequence determined. FIG. 3

Recombinants: In one embodiment, the present invention identifies and provides a means of deleting part or all of nucleotide sequences of human subgroup B adenovirus, including the E1 region, particularly the E1B region, and/or E3 regions. If desired heterologous or homologous nucleotide sequences encoding foreign genes or fragments thereof can be inserted to generate human adenovirus recombinants. By “deleting part of” the nucleotide sequence is meant using conventional genetic engineering techniques for deleting the nucleotide sequence of part of the E1 and/or E3 region.

Insertions are made by art-recognized techniques including, but not limited to, restriction digestion, nuclease digestion, ligation, kinase and phosphatase treatment, DNA polymerase treatment, reverse transcriptase treatment, and chemical oligonucleotide synthesis. Foreign nucleic acid sequences of interest are cloned into plasmid vectors such that the foreign sequences are flanked by sequences having substantial homology to a region of the adenovirus genome into which insertion is to be directed. These constructs are then introduced into host cells that are coinfected with the desired subgroup B virus. During infection, homologous recombination between these constructs and adenoviral genomes will occur to generate recombinant adenoviral vectors. If the insertion occurs in an essential region of the adenoviral genome, the recombinant adenoviral vector is propagated in a helper cell line which supplies the viral function that was lost due to the insertion.

A preferred adenoviral deletion is one in which all or part of the E1B region that encodes the oncoprotein, 55K, is removed. This deletion has the effect of producing a replication deficient subgroup B adenovirus. Similarly, by making select mutations in the E1B region it is possible to generate subgroup B adenovirues that are replication deficient. See, U.S. Pat. No. 6,080,578. Such replication deficient subgroup B adenovirus will be oncolytic for tumor cells that lack p53 function, and that primarily infect neoplastic cells by non-CAR mechanisms. Because CAR is present at high levels on liver cells and is often reduced on tumor cells, a subgroup B replication deficient adenovirus will have enhanced systemic activity in that it will not readily be taken up by the liver compared, for example, to subgroup C adenoviruses. Thus, it will also exhibit elevated levels of oncolytic activity when compared to subgroup C adenoviruses.

In an alternative embodiment of the invention, a recombinant subgroup B adenovirus can be constructed that comprises a deletion or mutation in an E1a locus that encodes an E1a oncoprotein protein, which causes the E1a protein to be substantially incapable of forming a complex with RB protein in infected cells. See, for example, U.S. Pat. No. 5,801,029.

The advantage of this type of recombinant subgroup B virus is its substantial incapacity to effectively sequester RB protein in infected non-neoplastic cells which results in the introduced recombinant adenovirus failing to express a replication phenotype in non-neoplastic cells. By contrast, neoplastic cells which lack a functional RB protein support expression of a replication phenotype by the introduced recombinant adenovirus which leads to ablation of the neoplastic cell by an adenoviral cytopathic effect.

In preferred variations of these embodiments, the recombinant subgroup B adenovirus comprises an E1 a locus encoding a mutant E1 a protein that lacks a domain capable of binding pRB (and/or the 300 kd-polypeptide and/or the 107 kD polypeptide) but comprises a functional E1 a domain capable of transactivation of adenoviral early genes. Additional variations of these embodiments include those where the recombinant adenovirus comprises a nonfunctional E1 a locus which is substantially incapable of expressing a protein that binds to and inactivates pRB In another embodiment, the invention provides compositions and methods for constructing, isolating and propagating E3-deleted recombinant adenoviral subgroup B (with or without insertion of heterologous sequences) at high efficiency. These include isolation of recombinant virus in suitable cell lines, expressing adenovirus E1 function or equivalent cell lines, and methods wherein recombinant genomes are constructed via homologous recombination in the appropriate host cells, the recombinant genomes obtained thereby are transfected into suitable cell lines, and recombinant virus is isolated from the transfected cells. See, for example, U.S. Pat. No. 6,492,169.

In one embodiment of the invention, a recombinant adenoviral subgroup B expression cassette can be obtained by cleaving the wild-type genome with one or more appropriate restriction enzyme(s) to produce a viral restriction fragment comprising; for example, E1, preferrably E1A that encodes the oncoprotein that binds pRB, or E1B that encodes the 55K protein that binds p53, or E3 region sequences, respectively. The viral restriction fragment can be inserted into a cloning vehicle, such as a plasmid, and thereafter at least one heterologous sequence (which may or may not encode a foreign protein) can be inserted into the chosen viral region with or without an operatively-linked eukaryotic transcriptional regulatory sequence. The recombinant expression cassette is contacted with a adenoviral subgroup B genome and, through homologous recombination in a suitable host cell, or other conventional genetic engineering methods, the desired recombinant is obtained.

Suitable host cells include any cell that will support recombination between an adenoviral subgroup B genome and a plasmid containing viral sequences, or between two or more plasmids, each containing viral sequences. Recombination may be performed in procaryotic cells, such as E. coli, while transfection of a plasmid containing a viral genome, to generate virus particles, is conducted in eukaryotic cells, preferably mammalian cells, more preferably 293 cells, and their equivalents. The growth of bacterial cell cultures, as well as culture and maintenance of eukaryotic cells and mammalian cell lines are procedures which are well-known to those of skill in the art.

One or more heterologous sequences can be inserted into one or more regions of an adenoviral subgroup B genome to generate a recombinant viral vector, limited only by the insertion capacity of the viral genome and ability of the recombinant viral vector to express the inserted heterologous sequences. Fusion proteins can be generated in this way. In general, adenovirus genomes can accept inserts of approximately 5% of genome length and remain capable of being packaged into virus particles. The insertion capacity can be increased by deletion of non-essential regions and/or deletion of essential regions whose function is provided by a helper cell line.

In one embodiment of the invention, insertion can be achieved by constructing a plasmid containing the region of the subgroup B adenoviral genome into which insertion is desired. The plasmid is then digested with a restriction enzyme having a recognition sequence in the viral portion of the plasmid, and a heterologous sequence is inserted at the site of restriction digestion. The plasmid, containing a portion of the viral genome with an inserted heterologous sequence, is co-transformed, along with an adenoviral genome or a linearized plasmid containing a adenoviral genome, into a bacterial cell (such as, for example, E. coli), wherein the adenoviral genome can be a full-length genome or can contain one or more deletions. Homologous recombination between the plasmids generates a recombinant adenoviral genome containing inserted heterologous sequences.

Deletion of adenoviral subgroup B sequences, to provide a site for insertion of heterologous sequences or to provide additional capacity for insertion at a different site, can be accomplished by methods well-known to those of skill in the art. For example, for sequences cloned in a plasmid, digestion with one or more restriction enzymes (with at least one recognition sequence in the viral insert) followed by ligation will, in some cases, result in deletion of sequences between the restriction enzyme recognition sites. Alternatively, digestion at a single restriction enzyme recognition site within the viral insert, followed by exonuclease treatment, followed by ligation will result in deletion of viral sequences adjacent to the restriction site. A plasmid containing one or more portions of the adenoviral genome with one or more deletions, constructed as described above, can be co-transfected into a bacterial cell along with an adenoviral subgroup B genome (full-length or deleted) or a plasmid containing either a full-length or a deleted viral genome to generate, by homologous recombination, a plasmid containing a recombinant viral genome with a deletion at one or more specific sites. Subgroup B viruses containing the deletion can then be obtained by transfection of mammalian cells with the plasmid containing the recombinant viral genome.

In one embodiment of the invention, insertion sites can be adjacent to and downstream (in the transcriptional sense) of the adenoviral promoters. Locations of promoters, and restriction enzyme recognition sequences for use as insertion sites, can be easily determined by one of skill in the art from the subgroup B adenoviral nucleotide sequence provided herein. Alternatively, various in vitro techniques can be used for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth., Enzymology 154:367-382) and PCR-mediated methods for insertion of longer sequences. See, for example, Zheng et al. (1994) Virus Research 31:163-186.

It is also possible to obtain expression of a heterologous sequence inserted at a site that is not downstream from an adenoviral subgroup B promoter, if the heterologous sequence additionally comprises transcriptional regulatory sequences that are active in eukaryotic cells.

The invention also provides adenoviral subgroup B regulatory sequences which can be used to regulate the expression of heterologous genes. A regulatory sequence can be, for example, a transcriptional regulatory sequence, a promoter, an enhancer, an upstream regulatory domain, a splicing signal, a polyadenylation signal, a transcriptional termination sequence, a translational regulatory sequence, a ribosome binding site and a translational termination sequence.

In another embodiment, the invention identifies and provides additional regions of the subgroup B adenoviral genomes (and fragments thereof) suitable for insertion of heterologous or homologous nucleotide sequences encoding foreign genes or fragments thereof to generate viral recombinants. In another embodiment, the clone subgroup B adenoviral genomes can be propagated as a plasmid and infectious virus can be rescued from plasmid-containing cells.

The presence of adenoviral nucleic acids can be detected by techniques known to one of skill in the art including, but not limited to, hybridization assays, polymerase chain reaction, and other types of amplification reactions. Similarly, methods for detection of proteins are well-known to those of skill in the art and include, but are not limited to, various types of immunoassay, ELISA, Western blotting, enzymatic assay, immunohistochemistry, etc. Various foreign genes or nucleotide sequences or coding sequences (prokaryotic, and eukaryotic) can be inserted in the adenovirus nucleotide sequences, e.g., DNA, in accordance with the present invention. An heterogenous nucleotide sequence can consist of one or more gene(s) of interest, and preferably of therapeutic interest. In the context of the present invention, a gene of interest can code either for cytokines, such as interferons and interleukins; lymphokines; negative selection agents (e.g. thymidine kinases), membrane receptors such as the receptors recognized by pathogenic organisms (viruses, bacteria or parasites), preferably by the HIV virus (human immunodeficiency virus); or genes coding for growth factors. This list is not restrictive, and other genes of interest may be used in the context of the present invention.

A gene of interest can be of genomic type, of complementary DNA (cDNA) type or of mixed type (minigene, in which at least one intron is deleted). It can code for a mature protein, a precursor of a mature protein, in particular a precursor intended to he secreted and accordingly comprising a signal peptide, a chimeric protein originating from the fusion of sequences of diverse origins, or a mutant of a natural protein displaying improved or modified biological properties. Such a mutant may be obtained by, deletion, substitution and/or addition of one or more nucleotide(s) of the gene coding for the natural protein, or any other type of change in the sequence encoding the natural protein, such as, for example, transposition or inversion.

A gene of interest may be placed under the control of elements (DNA control sequences) suitable for its expression in a host cell. Suitable DNA control sequences are understood to mean the set of elements needed for transcription of a gene into RNA (antisense RNA or mRNA) and for the translation of an mRNA into protein. Among the elements needed for transcription, the promoter assumes special importance. It can be a constitutive promoter or a regulatable promoter, and can he isolated from any gene of eukaryotic, prokaryotic or viral origin, and even adenoviral origin. Alternatively, it can be the natural promoter of the gene of interest. Generally speaking, a promoter used in the present invention may be modified so as to contain regulatory sequences. A variety of promoters may be used, including the HSV-1 TK (herpesvirus type 1 thymidine kinase) gene promoter, the adenoviral MLP (major late promoter), in particular of human adenovirus type 2, the RSV (Rous Sarcoma Virus) LTR (long terminal repeat), the CMV (Cytomegalovirus) early promoter, and the PGK (phosphoglycerate kinase) gene promoter, for example, permitting expression in a large number of cell types.

A promoter that can be advantagously applied to regulate the replication of subgroup B adenovirus recombinants, or the expression of genes therefrom, is an E2F promoter as described in U.S. patent application Ser. No. 09/714,409, or EPA 1230378.

Targeting of a recombinant subgroup B adenoviral vector to a particular cell type can be achieved by constructing recombinant hexon and/or fiber genes. The protein products of these genes are involved in host cell recognition; therefore, the genes can be modified to contain peptide sequences that will allow the virus to recognize alternative host cells.

It is also possible that only fragments of nucleotide sequences of genes can be used (where these are sufficient to generate a protective immune response or a specific biological effect) rather than the complete sequence as found in the wild-type organism. Where available, synthetic genes or fragments thereof can also be used. However, the present invention can be used with a wide variety of genes, fragments and the like, and is not limited to those set out above.

In some cases the gene for a particular antigen can contain a large number of introns or can be from an RNA virus, in these cases a complementary DNA copy (cDNA) can be used.

In order for successful expression of the gene to occur, it can be inserted into an expression vector together with a suitable promoter including enhancer elements and polyadenylation sequences. A number of eucaryotic promoter and polyadenylation sequences which provide successful expression of foreign genes in mammalian cells and how to construct expression cassettes, are known in the art, for example in U.S. Pat. No. 5,151,267, the disclosures of which are incorporated herein by reference. The promoter is selected to give optimal expression of immunogenic protein which in turn satisfactorily leads to humoral, cell mediated and mucosal immune responses according to known criteria.

The present invention also includes pharmaceutical compositions comprising a therapeutically effective amount of a recombinant human adenoviral subgroup B virus or vector derived therefrom prepared according to the methods of the invention, in combination with a pharmaceutically acceptable vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially) or by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

In another embodiment of the invention, E1 function (or the function of other viral regions which may be mutated or deleted in any particular viral vector) can be supplied (to provide a complementing cell line) by co-infection of cells with a virus which expresses the function that the vector lacks.

The invention also includes an expression system comprising a subgroup B adenovirus expression vector wherein a heterologous nucleotide sequence, e.g. DNA, replaces part or all of the E3 region, part or all of the E1 or E1B regions, part or all of the E2 region, part or all of the E4 region, part or all of the region between E4 and the right end of the genome, part or all of the late regions (L1-L7) and/or part or all of the regions occupied by penton genes. The expression system can be used wherein the foreign nucleotide sequences, e.g. DNA, is with or without the control of any other heterologous promoter.

The practice of the present invention in regard to gene therapy in humans is intended for the prevention or treatment of diseases including, but not limited to cancers, cardiovascular diseases, and the like. As applied to the treatement of cancer, the adenoviral vectors can be combined with chemotherapy. For the purposes of the present invention, the vectors, cells and viral particles prepared by the methods of the invention may be introduced into a subject either ex vivo, (i.e., in a cell or cells removed from the patient) or directly in vivo into the body to be treated. Preferably, the host cell is a human cell and, more preferably, is a lung, fibroblast, muscle, liver or lymphocytic cell or a cell of the hematopoietic lineage.

Adenoviruses of the invention may be formulated for therapeutic and diagnostic administration to a patient. For therapeutic or prophylactic uses, a sterile composition containing a pharmacologically effective dosage of adenovirus is administered to a human patient or veterinary non-human patient for treatment, for example, of a neoplastic condition. Generally, the composition will comprise about 10³ to 10¹⁵ or more adenovirus particles in an aqueous suspension. A pharmaceutically acceptable carrier or excipient is often employed in such sterile compositions. A variety of aqueous solutions can be used, e.g., water, buffered water, 0.4% saline, 0.3% glycine and the like. These solutions are sterile and generally free of particulate matter other than the desired adenoviral vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients which enhance infection of cells by adenovirus may be included.

Subgroup B adenoviruses of the invention, or the DNA contained therein, may also be delivered to neoplastic cells by liposome or immunoliposome delivery; such delivery may be selectively targeted to neoplastic cells on the basis of a cell surface property present on the neoplastic cell population (e.g., the presence of a cell surface protein which binds an immunoglobulin in an immunoliposome). Typically, an aqueous suspension containing the virions are encapsulated in liposomes or immunoliposomes. For example, a suspension of adenovirus virions can be encapsulated in micelles to form immunoliposomes by conventional methods (U.S. Pat. No. 5,043,164, U.S. Pat. No. 4,957,735, U.S. Pat. No. 4,925,661; Connor and Huang (1985) J. Cell Biol. 101: 582; Lasic DD (1992) Nature 355: 279; Novel Drug Delivery (eds. Prescott LF and Nimmo WS: Wiley, New York, 1989); Reddy et al. (1992) J. Immunol. 148: page 1585). Immunoliposomes comprising an antibody that binds specifically to a cancer cell antigen (e.g., CALLA, CEA) present on the cancer cells of the individual may be used to target virions, or virion DNA to those cells.

The compositions containing the present adenoviruses or cocktails thereof can be administered for therapeutic treatments of neoplastic disease. In therapeutic application, compositions are administered to a patient affected by the particular neoplastic disease, in an amount sufficient to cure or at least partially arrest the condition and its complications. An amount adequate to accomplish this is defined as a “therapeutically effective dose” or “efficacious dose.” Amounts effective for this use will depend upon the severity of the condition, the general state of the patient, and the route of administration.

Described below are examples of the present invention. These examples are provided only for illustrative purposes and are not intended to limit the scope of the present invention in any way. In light of the present disclosure, numerous embodiments within the scope of the claims will be apparent to those of ordinary skill in the art. The contents of the references cited in the specification are incorporated by reference herein.

### EXAMPLES

### Example 1

### Adenovirus 3 and 34 Genomic Sequences

Human subgroup B adenovirus types 3 and 34 (hereinafter also referred to as Ad3 or Ad 34, respectively) were obtained from the American Type Culture Collection (ATCC). The viruses were propagated in A549 cells, also available from the ATCC, and using standard infection and growth techniques. Both viruses were purified by cesium chloride gradient banding centrifugation.

Viral DNA was obtained from cesium chloride gradient-banded virus particles by lysing the virus particles in a solution consisting of: 10 mM Tris-HCl (pH8.0), SM EDTA, 0.6% SDS and 1.5 mg per ml of pronase (Sigma Corporation). The solution was at 37° C. Lysed particles were extracted twice with phenol/chloroform, and viral DNA was precipitated with ethanol. Purified viral DNAs were dissolved in distilled water and used for DNA sequencing.

Next, viral DNAs were subjected to limited digestion with Sau 3AI, followed by resolving the digested DNAs in a 1% agarose gel. Fragments between 0.8 kb and 1.2 kb in size were purified using a commercial DNA gel extraction kit (Qiagen Corporation), and subsequently cloned in Bam HI digested vector, pGem-7zf(+) (Promega Corporation).

Two hundred individual clones were sequenced using an automated sequencer, CEQ20000XL (Beckman), and using standard T7 and SP6 Sequencing primers. Contigs were constructed using SeqMan Software (DNAStar Inc.). Based on the constructed sequences, oligonucleotides were synthesized and primer walking was performed until all contiges were joined. Most regions were covered by at least 2 independent sequencings.

### Example 2

### Construction of E1B 55K Deleted Virus on Ad34 Backbone

Plasmid Construction

Vectors based on pGEM (Promega Corp.) were modified and used to clone, subclone the relevant nucleotide sequences. Plasmid construction was based on the fact that there is a unique NheI restriction site in the Ad34 genome at 6.5 KB from the left end. Plasmid construction began with the digest of the Ad34 genome (15 ug) with HindIII. Two fragments sized 2.2 Kb and 3.4 Kb were isolated on a 1% agarose gel and purified using Bio 101 Gene Clean Kit. The 2.2 Kb fragment was ligated into pGEM-7Z (Promega), that had been previously digested with HindIII. The construct was evaluated for the correct fragment and orientation by restriction mapping. This construct was called 2.2/pGEM-7Z. Next, the HindIII site near the NheI site in the 2.2/pGEM7Z construct was removed by digesting with NheI and ClaI, then filling in with Klenow and re-ligating. The first 1.4 Kb of the Ad34 genome, was generated by PCR (U.S. Pat. No. 4,683,202)

using PCR primers PO₄ Fwd (5′CATGAGCTCGCGGCCGCCATCATCAATAATATACCTTATAGA-3′) and Ad34-1370B (5′GGCTTAAGCTTCACAGGAA-3′), 1 ng genomic template DNA and Pfu DNA Polymerase (Stratagene). The PCR product was purified using QIAquick PCR Purification kit (QIAGEN), digested with SacI and HindIII, isolated on 1% agarose gel and purified with Bio 101 Gene Clean Kit. Purified 1.4 Kb fragments were ligated to the 2.2/pGEM-7Z that had been digested with SacI and HindIII, to create the 1.4/2.2/pGEM-7Z construct. The 3.4 Kb fragment was ligated into pGEM-9Z (Promega), that had been previously digested with HindIII. The construct was evaluated for the correct fragment and orientation by restriction mapping. As the E1B19K and E1B55K genes overlap, inactivation of the E1B55K gene was achieved by introducing a stop codon following the start site of E1B55K and a deletion of the sequence between the end of the E1B19K end and the rest of the E1B55K gene. The deleted region was replaced with a PmeI site. The mutagenesis of the E1B55K was performed using a two step PCR process with the 3.4/pGEM9Z construct. The product from the first step of the PCR was generated using PCR primers P02 fwd (5′-CCCTCCAGTGGAGGAGGCGGAGTAGGTTTAAACGGTGAGTATTGGGAAAAC TTGGGGT-3′), PO₃ Rev (5′-TAGCATAGGTCAGCGTTGAAGAAT-3′), 10ng 3.4/pGEM-9Z template DNA and Faststart DNA Polymerase (Roche). The second PCR step was generated using the PCR Primers P01 fwd (5′-ATAAATGGATCCCGCAGACTCATTTTAGCAGGGGATACGTTTTGGATTTCG-3′) and the product from the first PCR reaction, 10 ng 3.4/pGEM9Z template DNA and Faststart DNA Polymerase (Roche). The PCR product was purified using a QIAquick PCR Purification Kit (QIAGEN), digested with BsmBI and BamHI, isolated on a 2% agarose gel and purified with Bio101 GeneClean. The purified E1B55K deleted fragment was ligated into 3.4/pGEM9Z that had been previously digested with BsmBI and BamHI, to generate 3.4Δ55K/pGEM-9Z. To assemble the 1.4 Kb, 2.2 Kb fragment and the 3.4Δ55K fragment, the 3.4Δ55K/pGEM-9Z construct was digested with HindIII, the 3.4Δ55K fragment was isolated on a 1% agarose gel and purified with Bio 101 Gene Clean Kit. Purified 3.4Δ55K fragments were ligated into the 1.4/2.2/pGEM-7Z construct that had been digested with HindIII and treated with CIP to create the shuttle vector, SV13.

After sequencing the shuttle vector, SV13, it was discovered that there was a single point mutation in the 1.4 KB fragment, caused by an error in the PCR. To correct this error, a new 1.4 KB PCR product was generated using the same PCR conditions with the exception that a proofreading DNA polymerase (pfu from Stratagene) was used. Purified 1.4 Kb fragments were ligated to the SV13 shuttle vector that had been digested with NotI and BmgBI to generate the shuttle vector, SV2-5. This construct was verified by sequencing.

Virus Construction and Isolation

Subgroup B adenovirus type 34 (Ad34) (ATCC) TP DNA was made as described by S. Miyake, et. al. (PNAS 1996). To construct the Ad34AE1B55K virus, the SV2-5 construct was digested with NotI and NheI (8.5 ug), isolated on a 1% agarose gel and purified with QIAquick Gel Purification Kit (QIAGEN). 5 ug of this fragment was then ligated O/N at RT to 0.25 ug AD34-TP DNA that had been digested with NheI at 37° C. for 6 hours. The ligation mixture was transfected into HEK293 cells in DMEM supplemented with 2% FBS media in 60 mm dishes using the Mammalian Transfection Kit (Stratagene) as per the manufacturer's protocols. The transfection was incubated O/N at 37° C./3% CO₂ for 24 hours. Transfections were stopped after 24 hours by removing the media and replacing it with DMEM supplemented with 2% FBS, 2% L-Glutameine, 1% PS which were then incubated for 24 hours at 37° C./5% CO₂. The cells were overlaid with DMEM infection media containing 2% FBS, 2% L-Glutamine, 1% NEAA, 1% PS and 1.5% SeaPlaque agarose and fed every 2-3 days with fresh overlay media. Plaques were isolated, propagated on HEK293 cells and viral DNA was isolated using the QIAamp DNA Blood Kit (QIAGEN) as per the manufacturer's recommendations. Viruses were screened by PCR for the E1B55K deleted region using the following primers: SVfwd05(5′-GGAAGACCTTAGAAAGACTAGGC-3′) and PO₃ Rev(5′-TAGCATAGGTCAGCGTTGAAGAAT-3′) PCR was performed using Faststart DNA Polymerase (Roche) under the following cycling conditions: 1 cycle at 94° C. for 5 min, 25-30 cycles at 94° C. for 30 sec, 55° C. for 30 sec, and 72° C. for 30 sec-90 sec, and 1 cycle at 72° C. for 7 min and finally 4° C. indefinitely. Positive plaques were purified 4 rounds on 293/E4 cells (Microbix Biosystems Inc.). All virus isolates were screened by PCR for the E1B55K deletion and for internal Ad34 wildtype E1B55K sequence with primers: 3.4fwd03 (5′-GGGATGAAGTTTCTGTATTGC-3′) and 3.4rev12 (5′-GTCACATCTACACACACCGG-3′).

Ad34ΔE1B55K virus, the shuttle vector, SV2-5, are on deposit with the American Type Culture Collection with Accession Numbers ______ and ______, respectively.

Although the present invention has been described in some detail by way of illustration for purposes of clarity of understanding, it will be apparent that certain changes and modifications may be practiced within the scope of the claims.

1. A method for ablating neoplastic cells in a cell population, comprising the steps of:
contacting under infective conditions (1) a recombinant replication deficient subgroup B adenovirus lacking an expressed viral oncoprotein capable of binding a functional tumor suppressor gene product, with (2) a cell population comprising non-neoplastic cells containing said functional tumor suppressor gene product which forms a bound complex with a viral oncoprotein and neoplastic cells lacking said functional tumor suppressor gene product, thereby generating an infected cell population.

2. A method as described in claim 1 wherein said tumor suppressor is p53, or pRb.

3. A method according to claim 2, wherein said viral oncoprotein comprises an adenovirus E1b or E1A polypeptide.

4. A method as described in claim 3, wherein said subgroup B adenovirus is selected from the group consisting of types 3 or 34.

5. A method of treating a patient's cancer, comprsing the steps of administering to said patient a recombinant replication deficient subgroup B adenovirus lacking an expressed adenoviral oncoprotein capable of binding a functional tumor suppressor gene product.

6. A method as described in claim 5, wherein said subgroup B adenovirus is selected from the group consisting of types 3 or 34.

7. A method as described in claim 6, wherein said adenoviral oncoprotein comprises an E1b or E1A polypeptide.

8. A recombinant human adenovirus subgroup B type 3, comprising the nucleotide seqeunce shown in FIG. 1

9. A nucleotide sequence that hybridizes under stringent conditions to the nucleotide sequence shown in FIG. 1.

10. A recombinant human adenovirus subgroup B type 34, comprising the nucleotide seqeunce shown in FIG. 2

11. A nucleotide sequence that hybridizes under stringent conditions to the nucleotide sequence shown in FIG. 2.

12. A recombinant human adenovirus subgroup B type 3 as described in claim 8, comprising a mutation in the E1A and/or E1B regions that encode oncoproteins that bind pRb or p53, respectively, said mutation reducing or eliminating binding of said oncoproteins to pRb or p53, respectively.

13. A recombinant human adenovirus as described in claim 12, wherein said mutation is a deletion or point mutation.

14. A recombinant human adenovirus subgroup B type 34 as described in claim 10, comprising a mutation in the E1A and/or E1B regions that encode oncoproteins that bind pRb or p53, respectively, said mutation reducing or eliminating binding of said oncoproteins to pRb or p53, respectively.

15. A recombinant human adenovirus as described in claim 14, wherein said mutation is a deletion or point mutation.

16. The nucleotide sequences shown in FIG. 1 or 2, comprising the regions that encode the E1B 55K protein, or nucleotide sequences that hybridize to said sequences under stringent conditions.

17. The E1B 55K protein encoded by the nucleotide sequences shown in FIG. 1 or 2, or proteins encoded by nucleotide sequences that hybridize to said sequences under stringent conditions.
